SEQUENCE LISTING (4 sequences)

SEQ ID NO: 1 - protein, 181 aa, Artificial Sequence
  synthetic construct
HPIPDSSPLLQFGGQVRQRYLYTDDAQQTECHLEIREDGTVGCAADQSPESLLQLKALKPGVIQILGVKTSRFLCQRPDGALYGSLHFDPEACSFREDLLEDGYNVYQSEAHGLPLHLPGDKSPHRKPAPRGPARFLPLPGLPPALPEPPGILAPQPPDVGSSDPLRLVEPSQLLSPSFLG

SEQ ID NO: 2 - protein, 181 aa, HOMO SAPIENS
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 3 - protein, 180 aa, artificial sequence
  synthetic construct
HPIPDSSPLLQFGGQVRQRYLYTDDAQQTECHLEIREDGTVGCAADQSPESLLQLKALKPGVIQILGVKTSRFLCQRPDGALYGSLHFDPEACSFREDLKEDGYNVYQSEAHGLPLHLPGDKSPHRKPAPRGPARFLPLPGLPPALPEPPGILAPQPPDVGSSDPLRLVEPSQLRSPSFE

SEQ ID NO: 4 - protein, 181 aa, artificial sequence
  synthetic construct
HPIPDSSPLLQFGGQVRQRYLYTDDAQQTECHLEIREDGTVGCAADQSPESLLQLKALKPGVIQILGVKTSRFLCQRPDGALYGSLHFDPEACSFREXLXEDGYNVYQSEAHGLPLHLPGDKSPHRKPAPRGPARFLPLPGLPPALPEPPGILAPQPPDVGSSDPLRLVEPSQLXSPSFXX

(19) United States
(12) Patent Application Publication
(10) Pub. No.: US 2015/0141335 A1
(43) Pub. Date: May 21, 2015

(54) THERAPEUTIC USES OF FIBROBLAST GROWTH FACTOR 21 PROTEINS

(71) Applicant: Eli Lily and Company, Indianapolis, IN (US)

(72) Inventors: Yanfei Linda Ma, Carmel, IN (US); Armando Rafael Irizarry Rovira, Carmel, IN (US); Vincent Louis Reynolds, Greenfield, IN (US)

(73) Assignee: Eli Lilly and Company, Indianapolis, IN (US)

(21) Appl. No.: 14/399,037

(22) PCT Filed: May 9, 2013
(86) PCT No.: PCT/US13/40275

§ 371 Date: Nov. 5, 2014

Related U.S. Application Data
(60) Provisional application No. 61/646,974, filed on May 15, 2012.
(60) Provisional application No. 61/786,939, filed on Mar. 15, 2013.

(51) Int. Cl. A61K 38/18

(52) U.S. Cl. CPC A61K 38/1825
USPC 514/9.1

(57) ABSTRACT

The present invention relates to therapeutic uses of human fibroblast growth factor 21 (FGF21) proteins.

The present invention relates to therapeutic uses of human fibroblast growth factor 21 (FGF21) proteins.

FGF21 is a hormone that functions as an important metabolic regulator of glucose and lipid homeostasis. FGF21 promotes glucose uptake in adipocytes by up-regulating GLUT1 expression, a mechanism distinct from that of insulin. In diabetic rodents and monkeys, human FGF21 lowered fasting serum concentrations of glucose and reduced fasting serum concentrations of triglycerides, insulin and glucagon. Furthermore, in rodent models of diet induced obesity, FGF21 administration led to cumulative body weight loss in a dose-dependent manner. Thus, FGF21 has potential utility for the treatment of diabetes, obesity, dyslipidemia, and metabolic syndrome.

Bone is a complex tissue and is constantly undergoing a complex process of renewal and remodeling involving many different factors and substances. Products and/or byproducts of bone formation, and resorption in particular, represent reliable indicators of bone remodeling. Biochemical markers of bone turnover are substances in blood and urine that reflect the relative activity of osteoblasts and osteoclasts. Currently available markers of bone resorption include pyridinoline (PYR), deoxypyridinoline (DPD), N-telopeptides of type 1 collagen (NTX), and C-telopeptides of type 1 collagen (CTX-1). Pyridinolines are measured in urine, whereas telopeptides can be measured in urine or serum. The most common markers of bone formation are osteocalcin (OCN), bone alkaline phosphatase (bone ALP), and procollagen type 1 N-terminal propeptide (P1NP). Measurements of bone turnover biomarkers represent an integrated picture of skeletal metabolism.

It does not appear that FGF21 has been studied or investigated as a means to increase bone formation and/or bone deposition. In fact, there are reported studies which suggest that increased FGF21 levels are actually associated with boss loss. For example, Wei et al. describes increased levels of FGF21 activity as being associated with bone loss (Wei et al., Fibroblast growth factor 21 promotes bone loss by potentiating the effects of peroxisome proliferator-activated receptor γ, Proc Natl Acac Sci, February 2012, vol. 109, no. 8: 3143-3148). Wei et al. discloses that both genetic and pharmacologic FGF21 gain of function lead to a striking decrease in bone mass in mice. Furthermore, Wei et al. states that the FGF21 loss of function leads to a reciprocal high-bone-mass phenotype and that mechanistically FGF21 inhibits osteoblastogenesis and stimulates adipogenesis from bone marrow mesenchymal stem cells by potentiating the activity of peroxisome proliferator-activated receptor γ (PPAR-γ).

The present examples demonstrate that a FGF21 protein delivered in vivo results in an increase of the osteoid deposition in or on a bone. Furthermore, the present examples demonstrate in transgenic mice that overexpression of human FGF21 results in an increase of the mineral content of a bone, when normalized for body weight and compared to wild-type mice. Together these results illustrate that a FGF21 protein may be used in the treatment of hypo-ostosis and/or hypo-osteoidosis. Thus, an objective of the present invention is to provide novel therapeutic uses of FGF21 proteins.

The present invention provides a method of increasing bone formation and/or bone deposition in a patient, comprising administering to the patient an effective amount of the FGF21 protein of SEQ ID NO: 1.

Furthermore, the present invention provides a method of treating hypo-ostosis in a patient, comprising administering to the patient an effective amount of the FGF21 protein of SEQ ID NO: 1.

In a further aspect, the present invention provides a method of accelerating the healing of a bone fracture, orthotic procedure, prosthetics implant, dental implant, and/or spinal fusion in a patient, comprising administering to the patient an effective amount of the FGF21 protein of SEQ ID NO: 1.

In a further aspect, the present invention provides a method of increasing the mineral density of a bone in a patient, comprising administering to the patient an effective amount of the FGF21 protein of SEQ ID NO: 1.

Furthermore, the present invention provides a method of increasing the mineral content of a bone in a patient, comprising administering to the patient an effective amount of the FGF21 protein of SEQ ID NO: 1.

In a further aspect, the present invention provides a method of increasing the osteoid deposition in or on a bone in a patient, comprising administering to the patient an effective amount of the FGF21 protein of SEQ ID NO: 1.

Furthermore, the present invention provides a method of treating hypo-osteoidosis in a patient, comprising administering to the patient an effective amount of the FGF21 protein of SEQ ID NO: 1.

Furthermore, the present invention provides a method of delaying, slowing, and/or preventing bone loss in a patient, comprising administering to the patient an effective amount of the FGF21 protein of SEQ ID NO: 1.

In a further aspect, the present invention provides a method of preventing and/or treating osteoporosis in a patient, comprising administering to the patient an effective amount of the FGF21 protein of SEQ ID NO: 1.

Furthermore, the present invention provides a method of preventing and/or treating high risk of fracture due to poor bone quality and/or bone loss in a patient, comprising administering to the patient an effective amount of the FGF21 protein of SEQ ID NO: 1.

The present invention provides a use of the FGF21 protein of SEQ ID NO: 1 in the manufacture of a medicament for increasing bone formation and/or bone deposition.

In a further aspect, the present invention provides a use of the FGF21 protein of SEQ ID NO: 1 in the manufacture of a medicament for treating hypo-ostosis.

In a further aspect, the present invention provides a use of the FGF21 protein of SEQ ID NO: 1 in the manufacture of a medicament for accelerating the healing of a bone fracture, orthotic procedure, prosthetics implant, dental implant, and/or spinal fusion.

In a further aspect, the present invention provides a use of the FGF21 protein of SEQ ID NO: 1 in the manufacture of a medicament for increasing the mineral density of a bone.

Furthermore, the present invention provides a use of the FGF21 protein of SEQ ID NO: 1 in the manufacture of a medicament for increasing the mineral content of a bone.

Furthermore, the present invention provides a use of the FGF21 protein of SEQ ID NO: 1 in the manufacture of a medicament for increasing the osteoid deposition in or on a bone.

In a further aspect, the present invention provides a use of the FGF21 protein of SEQ ID NO: 1 in the manufacture of a medicament for treating hypo-osteoidosis.

In a further aspect, the present invention provides a use of the FGF21 protein of SEQ ID NO: 1 in the manufacture of a medicament for delaying, slowing, and/or preventing bone loss.

Furthermore, the present invention provides a use of the FGF21 protein of SEQ ID NO: 1 in the manufacture of a medicament for preventing and/or treating osteoporosis.

Furthermore, the present invention provides a use of the FGF21 protein of SEQ ID NO: 1 in the manufacture of a medicament for preventing and/or treating high risk of fracture due to poor bone quality and/or bone loss.

The present invention provides the FGF21 protein of SEQ ID NO: 1 for use in increasing bone formation and/or bone deposition.

In a further aspect, the present invention provides the FGF21 protein of SEQ ID NO: 1 for use in treating hypo-ostosis.

In a further aspect, the present invention provides the FGF21 protein of SEQ ID NO: 1 for use in accelerating the healing of a bone fracture, orthotic procedure, prosthetics implant, dental implant, and/or spinal fusion.

In a further aspect, the present invention provides the FGF21 protein of SEQ ID NO: 1 for use in increasing the mineral density of a bone.

Furthermore, the present invention provides the FGF21 protein of SEQ ID NO: 1 for use in increasing the mineral content of a bone.

Furthermore, the present invention provides the FGF21 protein of SEQ ID NO: 1 for use in increasing the osteoid deposition in or on a bone.

Furthermore, the present invention provides the FGF21 protein of SEQ ID NO: 1 for use in treating hypo-osteoidosis.

In a further aspect, the present invention provides the FGF21 protein of SEQ ID NO: 1 for use in delaying, slowing, and/or preventing bone loss.

In a further aspect, the present invention provides the FGF21 protein of SEQ ID NO: 1 for use in preventing and/or treating osteoporosis.

In a further aspect, the present invention provides the FGF21 protein of SEQ ID NO: 1 for use in preventing and/or treating high risk of fracture due to poor bone quality and/or bone loss.

The FGF21 protein of SEQ ID NO: 1 may be used, as described herein, as a single agent and/or in combination with another agent or agents that promotes bone mineralization, bone formation, and/or decreases bone resorption. Agents include, but are not limited to, FORTEO®, EVISTA®, FOSAMAX®, ACTONEL®, and BONIVA®, zolendronate, denosumab, blososumab, CDP7851/AMG 785.

The present invention provides a method of increasing bone formation and/or bone deposition in a patient, comprising administering to the patient an effective amount of the FGF21 protein of SEQ ID NO: 3.

Furthermore, the present invention provides a method of treating hypo-ostosis in a patient, comprising administering to the patient an effective amount of the FGF21 protein of SEQ ID NO: 3.

In a further aspect, the present invention provides a method of accelerating the healing of a bone fracture, orthotic procedure, prosthetics implant, dental implant, and/or spinal fusion in a patient, comprising administering to the patient an effective amount of the FGF21 protein of SEQ ID NO: 3.

In a further aspect, the present invention provides a method of increasing the mineral density of a bone in a patient, comprising administering to the patient an effective amount of the FGF21 protein of SEQ ID NO: 3.

Furthermore, the present invention provides a method of increasing the mineral content of a bone in a patient, comprising administering to the patient an effective amount of the FGF21 protein of SEQ ID NO: 3.

In a further aspect, the present invention provides a method of increasing the osteoid deposition in or on a bone in a patient, comprising administering to the patient an effective amount of the FGF21 protein of SEQ ID NO: 3.

Furthermore, the present invention provides a method of treating hypo-osteoidosis in a patient, comprising administering to the patient an effective amount of the FGF21 protein of SEQ ID NO: 3.

Furthermore, the present invention provides a method of delaying, slowing, and/or preventing bone loss in a patient, comprising administering to the patient an effective amount of the FGF21 protein of SEQ ID NO: 3.

In a further aspect, the present invention provides a method of preventing and/or treating osteoporosis in a patient, comprising administering to the patient an effective amount of the FGF21 protein of SEQ ID NO: 3.

Furthermore, the present invention provides a method of preventing and/or treating high risk of fracture due to poor bone quality and/or bone loss in a patient, comprising administering to the patient an effective amount of the FGF21 protein of SEQ ID NO: 3.

The present invention provides a use of the FGF21 protein of SEQ ID NO: 3 in the manufacture of a medicament for increasing bone formation and/or bone deposition.

In a further aspect, the present invention provides a use of the FGF21 protein of SEQ ID NO: 3 in the manufacture of a medicament for treating hypo-ostosis.

In a further aspect, the present invention provides a use of the FGF21 protein of SEQ ID NO: 3 in the manufacture of a medicament for accelerating the healing of a bone fracture, orthotic procedure, prosthetics implant, dental implant, and/or spinal fusion.

In a further aspect, the present invention provides a use of the FGF21 protein of SEQ ID NO: 3 in the manufacture of a medicament for increasing the mineral density of a bone.

Furthermore, the present invention provides a use of the FGF21 protein of SEQ ID NO: 3 in the manufacture of a medicament for increasing the mineral content of a bone.

Furthermore, the present invention provides a use of the FGF21 protein of SEQ ID NO: 3 in the manufacture of a medicament for increasing the osteoid deposition in or on a bone.

In a further aspect, the present invention provides a use of the FGF21 protein of SEQ ID NO: 3 in the manufacture of a medicament for treating hypo-osteoidosis.

In a further aspect, the present invention provides a use of the FGF21 protein of SEQ ID NO: 3 in the manufacture of a medicament for delaying, slowing, and/or preventing bone loss.

Furthermore, the present invention provides a use of the FGF21 protein of SEQ ID NO: 3 in the manufacture of a medicament for preventing and/or treating osteoporosis.

Furthermore, the present invention provides a use of the FGF21 protein of SEQ ID NO: 3 in the manufacture of a medicament for preventing and/or treating high risk of fracture due to poor bone quality and/or bone loss.

The present invention provides the FGF21 protein of SEQ ID NO: 3 for use in increasing bone formation and/or bone deposition.

In a further aspect, the present invention provides the FGF21 protein of SEQ ID NO: 3 for use in treating hypo-ostosis.

In a further aspect, the present invention provides the FGF21 protein of SEQ ID NO: 3 for use in accelerating the healing of a bone fracture, orthotic procedure, prosthetics implant, dental implant, and/or spinal fusion.

In a further aspect, the present invention provides the FGF21 protein of SEQ ID NO: 3 for use in increasing the mineral density of a bone.

Furthermore, the present invention provides the FGF21 protein of SEQ ID NO: 3 for use in increasing the mineral content of a bone.

Furthermore, the present invention provides the FGF21 protein of SEQ ID NO: 3 for use in increasing the osteoid deposition in or on a bone.

Furthermore, the present invention provides the FGF21 protein of SEQ ID NO: 3 for use in treating hypo-osteoidosis.

In a further aspect, the present invention provides the FGF21 protein of SEQ ID NO: 3 for use in delaying, slowing, and/or preventing bone loss.

In a further aspect, the present invention provides the FGF21 protein of SEQ ID NO: 3 for use in preventing and/or treating osteoporosis.

In a further aspect, the present invention provides the FGF21 protein of SEQ ID NO: 3 for use in preventing and/or treating high risk of fracture due to poor bone quality and/or bone loss.

The FGF21 protein of SEQ ID NO: 3 may be used, as described herein, as a single agent and/or in combination with another agent or agents that promotes bone mineralization, bone formation, and/or decreases bone resorption. Agents include, but are not limited to, FORTEO®, EVISTA®, FOSAMAX®, ACTONEL®, and BONIVA®, zolendronate, denosumab, blososumab, CDP7851/AMG 785.

The present invention provides a method of increasing bone formation and/or bone deposition in a patient, comprising administering to the patient an effective amount of the FGF21 protein, wherein the amino acid sequence is SEQ ID NO: 4.

Furthermore, the present invention provides a method of treating hypo-ostosis in a patient, comprising administering to the patient an effective amount of the FGF21 protein, wherein the amino acid sequence is SEQ ID NO: 4.

In a further aspect, the present invention provides a method of accelerating the healing of a bone fracture, orthotic procedure, prosthetics implant, dental implant, and/or spinal fusion in a patient, comprising administering to the patient an effective amount of the FGF21 protein, wherein the amino acid sequence is SEQ ID NO: 4.

In a further aspect, the present invention provides a method of increasing the mineral density of a bone in a patient, comprising administering to the patient an effective amount of the FGF21 protein, wherein the amino acid sequence is SEQ ID NO: 4.

Furthermore, the present invention provides a method of increasing the mineral content of a bone in a patient, comprising administering to the patient an effective amount of the FGF21 protein, wherein the amino acid sequence is SEQ ID NO: 4.

In a further aspect, the present invention provides a method of increasing the osteoid deposition in or on a bone in a patient, comprising administering to the patient an effective amount of the FGF21 protein, wherein the amino acid sequence is SEQ ID NO: 4.

Furthermore, the present invention provides a method of treating hypo-osteoidosis in a patient, comprising administering to the patient an effective amount of the FGF21 protein, wherein the amino acid sequence is SEQ ID NO: 4.

Furthermore, the present invention provides a method of delaying, slowing, and/or preventing bone loss in a patient, comprising administering to the patient an effective amount of the FGF21 protein, wherein the amino acid sequence is SEQ ID NO: 4.

In a further aspect, the present invention provides a method of preventing and/or treating osteoporosis in a patient, comprising administering to the patient an effective amount of the FGF21 protein, wherein the amino acid sequence is SEQ ID NO: 4.

Furthermore, the present invention provides a method of preventing and/or treating high risk of fracture due to poor bone quality and/or bone loss in a patient, comprising administering to the patient an effective amount of the FGF21 protein, wherein the amino acid sequence is SEQ ID NO: 4.

The present invention provides a use of the FGF21 protein, wherein the amino acid sequence is SEQ ID NO: 4 in the manufacture of a medicament for increasing bone formation and/or bone deposition.

In a further aspect, the present invention provides a use of the FGF21 protein, wherein the amino acid sequence is SEQ ID NO: 4 in the manufacture of a medicament for treating hypo-ostosis.

In a further aspect, the present invention provides a use of the FGF21 protein, wherein the amino acid sequence is SEQ ID NO: 4 in the manufacture of a medicament for accelerating the healing of a bone fracture, orthotic procedure, prosthetics implant, dental implant, and/or spinal fusion.

In a further aspect, the present invention provides a use of the FGF21 protein, wherein the amino acid sequence is SEQ ID NO: 4 in the manufacture of a medicament for increasing the mineral density of a bone.

Furthermore, the present invention provides a use of the FGF21 protein, wherein the amino acid sequence is SEQ ID NO: 4 in the manufacture of a medicament for increasing the mineral content of a bone.

Furthermore, the present invention provides a use of the FGF21 protein, wherein the amino acid sequence is SEQ ID NO: 4 in the manufacture of a medicament for increasing the osteoid deposition in or on a bone.

In a further aspect, the present invention provides a use of the FGF21 protein, wherein the amino acid sequence is SEQ ID NO: 4 in the manufacture of a medicament for treating hypo-osteoidosis.

In a further aspect, the present invention provides a use of the FGF21 protein, wherein the amino acid sequence is SEQ ID NO: 4 in the manufacture of a medicament for delaying, slowing, and/or preventing bone loss.

Furthermore, the present invention provides a use of the FGF21 protein, wherein the amino acid sequence is SEQ ID NO: 4 in the manufacture of a medicament for preventing and/or treating osteoporosis.

Furthermore, the present invention provides a use of the FGF21 protein, wherein the amino acid sequence is SEQ ID NO: 4 in the manufacture of a medicament for preventing and/or treating high risk of fracture due to poor bone quality and/or bone loss.

The present invention provides the FGF21 protein, wherein the amino acid sequence is SEQ ID NO: 4 for use in increasing bone formation and/or bone deposition.

In a further aspect, the present invention provides the FGF21 protein, wherein the amino acid sequence is SEQ ID NO: 4 for use in treating hypo-ostosis.

In a further aspect, the present invention provides the FGF21 protein, wherein the amino acid sequence is SEQ ID NO: 4 for use in accelerating the healing of a bone fracture, orthotic procedure, prosthetics implant, dental implant, and/or spinal fusion.

In a further aspect, the present invention provides the FGF21 protein, wherein the amino acid sequence is SEQ ID NO: 4 for use in increasing the mineral density of a bone.

Furthermore, the present invention provides the FGF21 protein, wherein the amino acid sequence is SEQ ID NO: 4 for use in increasing the mineral content of a bone.

Furthermore, the present invention provides the FGF21 protein, wherein the amino acid sequence is SEQ ID NO: 4 for use in increasing the osteoid deposition in or on a bone.

Furthermore, the present invention provides the FGF21 protein, wherein the amino acid sequence is SEQ ID NO: 4 for use in treating hypo-osteoidosis.

In a further aspect, the present invention provides the FGF21 protein, wherein the amino acid sequence is SEQ ID NO: 4 for use in delaying, slowing, and/or preventing bone loss.

In a further aspect, the present invention provides the FGF21 protein, wherein the amino acid sequence is SEQ ID NO: 4 for use in preventing and/or treating osteoporosis.

In a further aspect, the present invention provides the FGF21 protein, wherein the amino acid sequence is SEQ ID NO: 4 for use in preventing and/or treating high risk of fracture due to poor bone quality and/or bone loss.

The FGF21 protein of SEQ ID NO: 4 may be used, as described herein, as a single agent and/or in combination with another agent or agents that promotes bone mineralization, bone formation, and/or decreases bone resorption. Agents include, but are not limited to, FORTEO®, EVISTA®, FOSAMAX®, ACTONEL®, and BONIVA®, zolendronate, denosumab, blososumab, CDP7851/AMG 785.

The methods and uses of the present invention comprise a preferred FGF21 protein of SEQ ID NO: 4, wherein the FGF21 protein is selected from the group consisting of SEQ ID NO: 1 and SEQ ID NO: 3. The most preferred FGF21 protein is SEQ ID NO: 1.

The term “treating” (or “treat” or “treatment”) means slowing, stopping, reducing, or reversing the progression or severity of a sign, symptom, disorder, condition, or disease.

The term “preventing” (or “prevent” or “prevention”) refers to a decrease in the occurrence or severity of a sign, symptom, disorder, condition, or disease or decrease in the risk of acquiring a sign, symptom, disorder, condition, or disease or its associated signs and/or symptoms in a subject.

A “patient” is a human.

The term “effective amount” refers to the amount or dose of the FGF21 protein of SEQ ID NO: 1, SEQ ID NO: 3, and/or SEQ ID NO: 4 that provides the desired treatment upon single or multiple dose administration to a patient.

The following examples may be performed essentially as described below.

### EXAMPLE 1

### Study 1

Male and female mice [Crl:CD1 (ICR)] are individually housed in stainless steel wire-mesh bottomed cages and are permitted a minimum of 10 days for acclimation prior to the initiation of treatment. Environmental controls are set to provide a 12-hour light/dark cycle, a temperature of 19-25° C., and a relative humidity of 30-70%. The mice are provided with a certified-pellet commercial laboratory diet (Teklad 2014C) and drinking water ad libitum. The mice are approximately 10 weeks of age at the initiation of treatment. Prior to treatment initiation, all mice are weighed and assigned to treatment groups to ensure homogeneity of group means and variances for body weights. The treatment groups each consist of 10 male mice and 10 female mice which receive daily subcutaneous injections of 0 (vehicle control), 0.5, 60, or 500 mg/kg of FGF21 protein of SEQ ID NO: 1 in a dosing volume of 2 mL/kg (for the 0.5 mL/kg group) or 10 mL/kg (for the remaining groups). The FGF21 protein of SEQ ID NO: 1 is formulated in 10 mM sodium citrate, 150 mM sodium chloride, pH 7 in Sterile Water for Injection USP, and filtered through 0.22 millimeter polyvinylidene difluoride (PDVF) filters. The FGF21 protein of SEQ ID NO: 1 is administered by subcutaneous injection into the scapular or dorsal regions. The treatments continue for up to 14 days. Samples of bone (femur and sternum) are collected, fixed and preserved in 10% neutral buffered formalin, embedded in paraffin wax, sectioned, stained with hematoxylin and eosin, and examined by light microscopy.

### Study 2

Male and female rats (Sprague Dawley CD/IGS) are individually housed in stainless steel ventilated cages. Environmental controls are set to provide a 12-hour light/dark cycle, a temperature of 68-79° F., and a relative humidity of 30-70%. Except when interrupted for laboratory procedures, the rats are provided with Harlan Teklad Global Diet-Rodent 2014C and drinking water ad libitum. The rats are assigned to treatment groups based on body weight stratification and are approximately 7-9 weeks of age at the initiation of treatment. The treatment groups each consist of 5 male rats and 5 female rats which received daily subcutaneous injections of 0 (vehicle control), 0.5, 5, 50, or 500 mg/kg of FGF21 protein of SEQ ID NO: 1 in a dosing volume of 1 mL/kg (for the 0.5 and 5 mg/kg groups) or 10 mL/kg (for the remaining groups). The FGF21 protein of SEQ ID NO: 1 is formulated in 10 mM sodium citrate, 150 mM sodium chloride, pH 7 in Sterile Water for Injection USP, and filtered through 0.22 millimeter polyvinylidene difluoride (PDVF) filters. The FGF21 protein of SEQ ID NO: 1 is administered daily by subcutaneous injection into the dorsal scapular or dorsal lumbar regions for 14 days. Samples of bone (femur and sternum) are collected, fixed and preserved in 10% neutral buffered formalin, embedded in paraffin wax, sectioned, stained with hematoxylin and eosin, and examined by light microscopy.

Hyperosteoidosis is seen histologically as follows:
In Study 1, hyperosteoidosis occurred in the femurs of all mice examined from the 500 mg/kg group.
In Study 2, hyperosteoidosis occurred in the femurs and sternums of all rats in the 500 mg/kg group.

Hyperosteoidosis in the femur was characterized by an increased deposition of eosinophilic fibrillar matrix (osteoid) in the metaphyseal trabeculae and along the diaphysis of the femur. In the sternum, the finding was evident as deposition of osteoid along endosteal and/or periosteal surfaces. The increased deposition of osteoid is an early step in new bone formation.

### EXAMPLE 2

Male rats [Crl:CD(SD) Sprague-Dawley] are individually housed in polycarbonate bins containing appropriate bedding material. Environmental controls are set to provide a 12-hour light/dark cycle, a temperature of 19-25° C., and a relative humidity of 30-70%. Except when interrupted for laboratory procedures, the rats are provided with Harlan Teklad Global Diet-Rodent 2014C and drinking water ad libitum. The rats are assigned to treatment groups based on body weight stratification and are approximately 12-13 weeks of age at the initiation of treatment. The treatment groups each consist of 10 male rats per timepoint which receive daily subcutaneous injections of 0 (vehicle control), 0.2, 2, 20, or 250 mg/kg of FGF21 protein of SEQ ID NO: 1 in a dosing volume of 5 mL/kg (for the 0, 20, and 250 mg/kg groups) or 0.4 mL/kg (for the remaining groups). The FGF21 protein of SEQ ID NO: 1 is formulated in 10 mM sodium citrate, 150 mM sodium chloride, pH 7 in Sterile Water for Injection, and filtered through 0.22 millimeter polyvinylidene difluoride (PDVF) filters. The FGF21 protein of SEQ ID NO: 1 is administered daily by subcutaneous injection into the dorsal scapular or dorsal lumbar regions for up to 28 days. Rats from various groups are evaluated after designated timepoints (i.e., after receiving 3, 7, 14, or 28 daily doses). Serum and urine samples are obtained and concentrations of biomarkers of bone formation (e.g., OCN) and bone resorption (e.g., CTX-1) are determined Samples of bone are collected, fixed and preserved in 10% neutral buffered formalin, embedded in paraffin wax, sectioned, stained with hematoxylin and eosin, and examined by light microscopy.

Increases in serum concentrations of OCN (a bone formation biomarker) arose after 3 daily doses at dose levels of 20 mg/kg or greater. The OCN concentrations remained elevated for the duration of the 28-day treatment phase. Decreases in urinary concentrations of CTX-1 (a bone resorption biomarker) also were seen at doses of 2 mg/kg or greater. Hyperosteoidosis, defined as an excess of pale staining osteoid, was seen histologically in multiple bones after as few as 3 daily doses and increased in magnitude with longer durations of treatment. After 28 days of treatment, hyperosteoidosis was observed at doses of 20 mg/kg or greater. The bones affected included femur, tibia, sternum, and vertebra. The increased deposition of osteoid is an early step in new bone formation. After a 1 month or 1.5 month reversibility period, the excess osteoid that was deposited during the dosing phase was mineralized and/or remodeled. The femur of some rats had increased cortical thickness compared to control rats.

### EXAMPLE 3

The skeletal phenotype and biomarkers of bone formation and resorption in 11-12 weeks old, male transgenic mice overexpressing human FGF21 are compared to wild-type mice. These evaluations also include FGF21 knock-out (KO) mice in which the endogenous murine FGF21 gene is inactivated. Skeletal phenotypes are assessed using micro Computed tomography (CT) scans of the lumbar vertebrae (LV), the proximal tibial metaphysis (PTM), and 2 points on the tibial diaphysis (TX1 and TX2). Bone formation biomarkers are P1NP and OCN, whereas the bone resorption biomarker is CTX-1. See: Ma et al., Teriparatide [rhPTH (1-34)], But Not Strontium Ranelate, Demonstrated Bone Anabolic Efficacy in Mature, Osteopenic, Ovariectomized Rats, Endocrinology 2011, 152: 1767-1778.

The micro CT bone scan results demonstrated that, when normalized for body weight, mean bone area values in the FGF21 transgenic mice were increased at all points measured (LV, PTM, TX1, and TX2), with the increases being statistically significant at LV and TX2. In contrast, bone area (adjusted for body weight) was decreased in the FGF21 KO mice at all points measured, with the decreases being statistically significant at the PTM and TX1. Mean bone mineral content values (normalized for body weight) in the FGF21 transgenic mice were increased for trabecular bone in LV and for cortical bone at TX2, with the change reaching statistical significance at TX2. Conversely, mean bone mineral content (normalized for body weight) was decreased in the FGF21 KO mice, with the changes being statistically significant for trabecular bone in the PTM and cortical bone at TX1. In addition, bone formation biomarkers (P1NP and OCN) were increased at 3 and 4 months age, then returned to age matched wild-type levels by 5 months of age and the bone resorption biomarker (CTX-1) was slightly, but significantly decreased at 3 month age, then returned to age matched wild-type levels by 5 months of age in the FGF21 transgenic mice. P1NP was statistically significantly decreased in the FGF21 KO mice at 3 months of age, then the value was not different from that was in wild-type mice. OCN and CTX-1 values in the FGF21 KO mice did not differ significantly from wild-type levels between 3-6 months of ages.

### EXAMPLE 4

3T3-L1-βKlotho fibroblasts are generated from 3T3-L1 fibroblasts by retroviral transduction of a CMV-driven mammalian expression vector containing the coding sequence of wild type mouse βKlotho and a blasticidin resistance marker. Blasticidin-resistant cells are selected after growth for 14 days in the presence of 15 μM blasticidin, and βKlotho protein expression is verified by immunoblot with an anti-βKlotho antibody. The 3T3-L1-βKlotho fibroblasts are maintained in Dulbecco's Modified Eagle Medium (DMEM) with 10% calf serum, and 15 μM blasticidin until plated for experimental use.

For glucose uptake, 3T3-L1-βKlotho fibroblasts are plated at 20,000 cells/well in 96-well plates and incubated for 48 hours in DMEM with 10% calf serum. The cells are incubated for 3 hours in DMEM with 0.1% bovine serum albumin (BSA) with or without a FGF21 protein of interest, followed by 1 hour incubation in Krebs-Ringer phosphate (KRP) buffer (15 mM Hepes, pH 7.4, 118 mM NaCl, 4.8 mM KCl, 1.2 mM MgSO₄, 1.3 mM CaCl₂, 1.2 mM KH₂PO₄, 0.1% BSA) containing 100 μM 2-deoxy-D-(¹⁴C) glucose with or without a FGF21 protein. Non-specific binding is determined by incubation of select wells in Krebs-Ringer bicarbonate/Hepes (KRBH) buffer containing 1 mM 2-deoxy-D-(¹⁴C) glucose. The reaction is terminated by addition of 20 μM cytochalasin B to the cells and glucose uptake is measured using a liquid scintillation counter.

The in vitro potency (EC₅₀) of the FGF21 protein of SEQ ID NO: 1 in the 3T3-L1-βKlotho fibroblast glucose uptake assay was 0.026 nM.

### EXAMPLE 5

### Construction of 293-βKlotho-SRE luc Reporter Cells

HEK-293 cells (human embryonic kidney cells) are cultured at 37° C., 5% CO₂ in growth medium (GM) containing 10% fetal bovine serum (FBS) in Dulbecco's modified Eagle's medium. Cells are co-transfected with a plasmid containing a CMV promoter driven human βKlotho expression cassette and a plasmid containing a Serum Response Element (SRE) driven luciferase expression cassette. The βKlotho expression plasmid also contains an SV40 promoter driven neomycin phosphotransferase expression cassette to confer resistance to the aminoglycoside antibiotic G418. Transfected HEK-293 cells are selected with 600 μg/mL of G418 to select for cells where the transfected plasmids have been integrated into the genome. Selected cells are cloned by dilution and tested for an increase in luciferase production at 24 hours post addition of FGF21. The clone demonstrating the largest FGF21 dependant increase in luciferase is chosen as the cell line used to measure relative FGF21 proteins activity.

### 293-βKlotho-SRE Luc FGF21 Activity Assay:

293-βKlotho-SRE luc cells are rinsed and placed into CD 293 suspension culture media (Invitrogen). Cells are grown in suspension overnight at 37° C., 6% CO₂, 125 rpm. Cells are counted, pelleted by centrifugation, and re-suspended in CD 293 media containing 0.1% BSA. Cells are placed in white 96 well plates at 25,000 cells per well. A four-fold serial dilution in CD 293/0.1% BSA is prepared for each FGF21 protein to generate eight dilutions with final concentrations from 100 nM to 0.006 nM. Dilutions are added to cells in triplicate and incubated for 16-20 hours at 37° C., 5% CO₂. Luciferase level is determined by the addition of an equal volume of OneGlo™ luciferase substrate (Promega) and measuring relative luminescence. Data are analyzed using a four parameter logistic model (XLfit version 5.1) to fit the curves and determine EC₅₀.

The in vitro potency (EC₅₀) of the FGF21 protein of SEQ ID NO: 1 in the human 293 cell-βKlotho-SRE luc assay was 0.25 nM.

### Sequences

FGF21 protein
SEQ ID NO: 1
HPIPDSSPLLQFGGQVRQRYLYTDDAQQTECHLEIREDGTVGCAADQS
PESLLQLKALKPGVIQILGVKTSRFLCQRPDGALYGSLHFDPEACSFR
EDLLEDGYNVYQSEAHGLPLHLPGDKSPHRKPAPRGPARFLPLPGLPP
ALPEPPGILAPQPPDVGSSDPLRLVEPSQLLSPSFLG
Wild Type FGF21 (Homo Sapiens)
SEQ ID NO: 2
HPIPDSSPLLQFGGQVRQRYLYTDDAQQTEAHLEIREDGTVGGAADQS
PESLLQLKALKPGVIQILGVKTSRFLCQRPDGALYGSLHFDPEACSFR
ELLLEDGYNVYQSEAHGLPLHLPGNKSPHRDPAPRGPARFLPLPGLPP
ALPEPPGILAPQPPDVGSSDPLSMVGPSQGRSPSYAS
FGF21 protein
SEQ ID NO: 3
HPIPDSSPLLQFGGQVRQRYLYTDDAQQTECHLEIREDGTVGCAADQSP
ESLLQLKALKPGVIQILGVKTSRFLCQRPDGALYGSLHFDPEACSFRED
LKEDGYNVYQSEAHGLPLHLPGDKSPHRKPAPRGPARFLPLPGLPPALP
EPPGILAPQPPDVGSSDPLRLVEPSQLRSPSFE
Consensus FGF21 protein
SEQ ID NO: 4
HPIPDSSPLLQFGGQVRQRYLYTDDAQQTECHLEIREDGTVGCAADQSP
ESLLQLKALKPGVIQILGVKTSRFLCQRPDGALYGSLHFDPEACSFRE
X₁LX₂EDGYNVYQSEAHGLPLHLPGDKSPHRKPAPRGPARFLPLPGLP
PALPEPPGILAPQPPDVGSSDPLRLVEPSQLX₃SPSFX₄X₅
X₁ is L or D
X₂ is L or K
X₃ is R or L
X₄ is L or E
X₅ is G or is absent

We claim:

1-22. (canceled)

23. A method of treating or preventing a disease or condition in a patient comprising administering the FGF21 protein of SEQ ID NO: 1 to a patient in need thereof, wherein the disease or condition is selected from the group consisting of hypo-ostosis, hypo-osteoidosis, osteoporosis, and high risk of fracture due to poor bone quality and/or bone loss.

24. A method of increasing bone formation, bone deposition, bone mineral density, bone mineral content, bone mineral density, and/or osteoid deposition in or on a bone in a patient, comprising administering the FGF21 protein of SEQ ID NO: 1 to the patient.

25. A method of accelerating the healing of a bone fracture, orthotic procedure, prosthetics implant, dental implant, and/or spinal fusion comprising administering the FGF21 protein of SEQ ID NO: 1.

26. A method of delaying, slowing, and/or preventing bone loss comprising administering the FGF21 protein of SEQ ID NO: 1.
